(19) United States
(12) Patent Application Publication
(10) Pub. No.: US 2026/0268505 A1
(43) Pub. Date: Sep. 10, 2026

(54) MOTION DETECTION BASED ON THREE-DIMENSIONAL IMAGE DATA

(71) Applicant: Siemens Healthineers AG, Forchheim (DE)

(72) Inventors: Michael Manhart, Fürth (DE); Alexander Preuhs, Erlangen (DE); Manuela Goldmann, Nürnberg (DE)

(21) Appl. No.: 19/555,485

(22) Filed: Mar. 3, 2026

(30) Foreign Application Priority Data
Mar. 10, 2025 (DE) 10 2025 109 000.9

(51) Int. Cl. G06T 7/246; G06T 7/73; G06T 15/00

(52) U.S. Cl. CPC G06T 7/248; G06T 7/74; G06T 15/00; G06T 2200/04; G06T 2207/20081

(57) ABSTRACT

To train a machine learning model, two-dimensional training projection images of an object are obtained. Three-dimensional training image data is reconstructed as a function of the plurality of training projection images and a predefined virtual motion of the object, wherein each training projection image is assigned a motion state according to the virtual motion. An effective angle in a target value range different from the output value range is calculated for each angulation angle. By applying the MLM to the three-dimensional training image data, a measure for a motion of the object is predicted for a predefined angle in the target value range. A value of a predefined loss function is calculated as a function of the predicted measure and the motion states of training projection images whose effective angles correspond to the predefined angle. The MLM is updated as a function of the value of the loss function.

The present patent document claims the benefit of German Patent Application No. 10 2025 109 000.9, filed Mar. 10, 2025, which is hereby incorporated by reference in its entirety.

### TECHNICAL FIELD

The present disclosure relates to a computer-implemented training method for training a machine learning model (MLM) for motion detection based on three-dimensional image data, wherein a plurality of two-dimensional training projection images representing an object are obtained and three-dimensional training image data is reconstructed as a function of the plurality of training projection images. The disclosure further relates to a corresponding computer-implemented method for motion detection, to a data processing system for performing such computer-implemented methods, to an imaging apparatus having such a data processing system, and to a corresponding computer program product.

### BACKGROUND

In medical imaging, patient movements represent a major challenge. In particular, such movements also affect imaging methods in which projection images having different projection directions are used in order to reconstruct three-dimensional image data, for instance X-ray-based imaging methods such as computed tomography (CT), cone beam computed tomography (cone beam CT (CBCT)) or tomosynthesis methods.

Motion-compensated reconstruction techniques may reduce motion artifacts by estimating or assuming a model of the patient movement and taking the model into account in the reconstruction. The reliability or accuracy of the model is crucial in order to avoid qualitatively unsatisfactory reconstructions and/or the need to repeat the scan.

In the publication by A. Preuhs et al., “Appearance learning for image-based motion estimation in tomography,” IEEE Transactions on Medical Imaging, 39 (11), 3667-3678 (2020), a deep autofocus (DAF) method for motion-compensated reconstruction is described in which a motion model is estimated by optimization of a learned image quality metric (IQM). The learned IQM is based on an artificial neural network (ANN) that is trained to predict a reprojection error (RPE) for slice images reconstructed from CBCT data. The RPE is then minimized in the course of an iterative reconstruction in order to compensate for the motion.

One difficulty lies in using such approaches for different image acquisition trajectories of the imaging device used, wherein the angulation angle range covered during the image acquisition is particularly relevant in this case. In a generic setup, the angulation angle range may have a width of more than 180°, for example, 220°. In this case, it is not easily possible for the ANN to differentiate between two angulation angles that are different by 180°. Another example would be situations in which the angulation angle range is shifted compared to a standard range, for instance due to limited flexibility in terms of patient positioning and support. If the standard range is given, for example, by [−20°, 200°], i.e., symmetrically about 90°, then the shifted angulation angle range may be [30°, 250°] if the object to be imaged is rotated through 50° relative to the standard orientation. Such situations may result in the trained ANN being unable to reliably determine the respective measure for the motion of the object, for example, the RPE.

The publication by N. Hansen, “The CMA evolution strategy: A tutorial,” (arXiv: 1604.00772) describes the CMA-ES optimization method.

In the publication by Z. Liu et al., “Swin transformer: Hierarchical vision transformer using shifted windows,” Proceedings of the IEEE/CVF international conference on computer vision, 10012-10022, a neural network referred to as a Swin transformer is described.

### SUMMARY AND DESCRIPTION

It is an object of the present disclosure to increase the reliability of the prediction of a measure for the motion of an object during the generation of projection images by an MLM.

The scope of the present disclosure is defined solely by the appended claims and is not affected to any degree by the statements within this summary. The present embodiments may obviate one or more of the drawbacks or limitations in the related art.

The disclosure is based on the idea of transforming the output value range of the angulation angle into a target value range having a width of max. 180° and training or using the MLM for predicting the measure for the motion of the object with respect to an angle in the target value range.

According to an aspect, a computer-implemented training method for training a machine learning model, MLM, for motion detection based on three-dimensional image data is disclosed. A plurality of two-dimensional training projection images representing an object are obtained, each training projection image of the plurality of training projection images being assigned an angulation angle within an output value range. Three-dimensional training image data is reconstructed as a function of the plurality of training projection images and a predefined virtual motion of the object, in particular a virtual motion of the object during the generation of the plurality of training projection images, each training projection image of the plurality of training projection images being assigned a motion state according to the virtual motion. An effective angle in a target value range that is different from the output value range is calculated for each angulation angle of the plurality of training projection images by a predefined mapping rule, a width of the target value range amounting to a maximum of 180°. By applying the MLM, in particular the MLM in the untrained or partially trained state, to the three-dimensional training image data, a measure for a motion of the object is predicted for a predefined angle in the target value range. A value of a predefined loss function is calculated as a function of the predicted measure and the motion states of all those training projection images of the plurality of training projection images whose effective angle corresponds to the predefined angle, in particular corresponds according to the mapping rule. The MLM is updated as a function of the value of the loss function.

Unless stated otherwise, all the acts of the computer-implemented training method may be performed by a data processing system that includes at least one data processing device. In particular, the at least one data processing device is configured or adapted to perform the acts of the computer-implemented training method. For this purpose, the at least one data processing device may store a computer program containing commands that, when they are executed by the at least one data processing device, cause the at least one data processing device to perform the computer-implemented training method. The computer-implemented training method may also be implemented entirely or partly in hardware. The terms “data processing system” and “at least one data processing device” may be used interchangeably here and in the following. This also applies to corresponding terms derived therefrom.

For the case in which the at least one data processing device contains two or more data processing devices, certain acts performed by the at least one data processing device may also be understood in the sense that different data processing devices perform different acts or different parts of an act. In particular, it is not necessary for each data processing device to perform the acts. In other words, the acts may be performed distributed over the two or more data processing devices.

An MLM, in particular a trained MLM, may be able to mimic cognitive functions that bring human beings into contact with thought processes of other human beings. In particular, by training based on training data, the MLM may be capable of adapting to new situations and of detecting and extrapolating patterns. Another term for a trained MLM is a “trained function.” A trained MLM may also be referred to as an algorithm trained by machine learning or as a model trained by machine learning or as a function trained by machine learning. An MLM may be implemented in software and/or hardware.

The parameters of an MLM may be adapted or updated by training. In particular, supervised training, semi-supervised training, unsupervised training, reinforcement learning, and/or active learning may be used in this case. In addition, representation learning, which is also referred to as feature learning, may also be used. In particular, the parameters of the MLMs may be adapted iteratively by multiple acts of the training. In particular, a certain loss function, which is also referred to as a cost function, may be minimized in the training. When training an artificial neural network (ANN), the backpropagation algorithm in particular may be used.

An MLM may contain an ANN, a support vector machine, a decision tree, and/or a Bayesian network, and/or the MLM may be based on k-means clustering, Q-learning, genetic algorithms, and/or association rules. In particular, an ANN may be or contain a deep neural network, a convolutional neural network (CNN), or a convolutional deep neural network. Furthermore, an ANN may be an adversarial network, a deep adversarial network, and/or a generative adversarial network (GAN).

The applying the MLM to the training image data and consequently the prediction of the measure for the motion may, where appropriate, be performed for further angles in the target value range, for example, for all angles in the target value range. It is also possible, in one act, i.e., by one-time application of the MLM to the training image data, to predict the measure for the motion for multiple, in particular all, angles in the target value range. In these cases, the value of the loss function may be calculated as a function of all of the thus predicted measures for the motion and of the corresponding motion states.

In particular, the measure for the motion may be predicted in each case for every angle of a plurality of angles in the target value range by applying the MLM to the training image data and a corresponding term of the loss function may be calculated as a function of the predicted measure and the motion states of all those training projection images whose effective angle corresponds to the respective angle. The value of the loss function is then calculated as a function of all of the thus calculated terms.

The described acts of the computer-implemented training method may correspond to just one training iteration. They may therefore be repeated in particular through variation of the virtual motion and/or through variation of the plurality of two-dimensional training projection images. The iterations may be performed until, for example, a predefined convergence criterion or abort criterion is met, e.g., the value of the loss function is less than a predefined limit value.

The training projection images may be simulated projection images, in particular simulated X-ray projection images. However, the training projection images may also be real projection images generated by an imaging device, in particular an X-ray-based imaging device. The X-ray-based imaging device may be a computed tomography device (CT device) or a cone-beam CT device (CBCT: cone-beam computed tomography), for example, a C-arm X-ray device, and so forth.

A per se known loss function may be used as the loss function, reference being made in particular here to the publication by Preuhs et al. cited in the introduction. The loss function includes in particular a term that quantifies a deviation of the predicted measure for the motion from a reference measure expected according to the motion states according to the virtual motion, or which is dependent on such a deviation. Since in principle multiple training projection images or angulation angles may be characterized by the same effective angle in the target value range, the deviation from the reference measure may also be determined for all angulation angles coming into consideration and the loss function may be dependent on all these deviations or only on the maximum value of these deviations, and so forth.

For example, the reference measure may correspond to a reprojection error according to the virtual motion and the loss function may be dependent on a deviation of the predicted measure for the motion from the reprojection error. In this context, the significance of a reprojection error is accorded to the predicted measure for the motion only in the course of the training, especially as the MLM by definition is updated in such a way that it may predict the reprojection error more and more accurately. This may also be applied to other measures for quantifying the motion apart from the reprojection error.

The updating of the MLM as a function of the value of the loss function may be accomplished with the aid of known algorithms, for example, a backpropagation algorithm.

The training projection images may be generated in particular based on raw data that is generated or captured or acquired by the imaging device. The generation of the raw data as well as the generation of the training projection images are in each case not necessarily part of the computer-implemented method, but rather the computer-implemented method may be positioned downstream of the generation of the projection images. However, each embodiment of the computer-implemented training method results in a corresponding embodiment of a training method that is not necessarily purely computer-implemented in that corresponding acts for generating the training projection images are incorporated.

The object is in particular a patient or a part of the body of the patient or a phantom or a part of the phantom. The motion of the object is to be understood here and in the following in particular as rigid motion, i.e., inflexible motion, or approximately rigid motion. The motion of the object may be construed as relative to a component of the imaging device, e.g., relative to a detector or a radiation source of the imaging device. A motion of the object may be considered as equivalent to a motion of the component of the imaging device.

The virtual motion of the object corresponds in particular to a simulated assumed motion of the object during the generation of the plurality of two-dimensional training projection images, even though the motion did not actually take place. In other words, the movement of the component of the imaging device relative to the object corresponded at least approximately to a desired trajectory or desired motion of the component of the imaging device. The virtual motion may be described by an arbitrary deviation, possibly limited by predefined physical and/or technical boundary conditions, from the desired trajectory. The motion state according to the virtual motion may therefore be given by the deviation of the spatial location, i.e., position and/or orientation, of the component of the imaging device relative to the object in relation to the corresponding location according to the desired trajectory. It is also possible to use other reference systems.

A known image reconstruction method may be used for the reconstruction of the three-dimensional training image data, for example, a method in which the reconstruction is based on a motion model of an estimated or otherwise determined movement of the object, as is explained, for example, in the publication by Preuhs et al. The desired trajectory may be used as a basis during the training of the MLM.

As a result of the reconstruction, a three-dimensional reconstructed volume, also referred to as a reconstruction, is therefore generated from the plurality of two-dimensional training projection images. For a predefined three-dimensional voxel grid, for example, the reconstruction, in this case, contains a corresponding voxel value for each voxel of the voxel grid, wherein the voxel values may correspond to attenuation values relating to the attenuation of X-ray radiation or like radiation that is used. The three-dimensional training data may contain the entire reconstruction or one or more parts thereof, for example, in the form of one or more slices, also referred to as slice images or slice acquisitions, having a specific slice thickness.

An angulation angle corresponds in particular to an angle that is included by a connecting line from a position of the component of the imaging device, for example, the radiation source, to the isocenter of the imaging device with a reference line, for example, a horizontal line. The angulation angles of the training projection images are therefore produced in particular from the desired trajectory and are therefore predefined. In certain examples, the output value range is predefined and discretized, for example, in acts having an increment of 0.1°, 0.5°, or 1°, such that the angulation angles are different only by multiples of the increment. The set of different angulation angles may completely fill the discretized output value range such that, in other words, at least one of the training projection images has a corresponding angulation angle for each discrete value in the output value range.

The mapping rule assigns an angle in the target value range to each angle in the output value range. The output value range is given by [symbol] [α [symbol] ₀, α₁] or by a corresponding set of discrete values. The width of the output value range is then given by A=α₁−α₀. The target value range is given by [symbol] [β [symbol] ₀, β₁] or by a corresponding set of discrete values. The width of the target value range is then given by B=β₁−β₀. Since the output value range differs from the target value range, α₀≠β₀ and/or α₁≠β₁ applies.

Since 0<B≤180° applies, whereas this is not analogously the case for A, in particular 0<B≤360°, the mapping rule is not necessarily injective. In other words, a value in the output value range cannot necessarily be determined uniquely for a given angle in the target value range. Since the mapping rule is known, however, it may be determined for a given angle in the target value range which angle or which angles in the output value range are mapped thereto.

Once the training of the MLM is completed, it may be applied to corresponding three-dimensional image data instead of to the training image data in order to predict the measure for the motion for a further object represented by the image data. This may be done as an end in itself, for example, in order to enable certain artifacts in the three-dimensional image data to be better interpreted based thereon. However, the predicted measure may also be used or processed further, for example, in order to estimate an actual motion of the further object, in which case it is in particular advantageous for this purpose if the measure for the motion is determined for all angles in the target value range.

Therefore, a unique assignability of a prediction of the measure of the motion to precisely one angle in the output value range is dispensed with and instead the prediction of the measure for the motion is performed for an effective angle in the target value range. This is advantageous since the target value range is max. 180° wide and therefore no geometric ambiguity occurs in the target value range. If the output value range has a width of more than 180°, then pairs of angulation angles of the training projection images exist that differ by 180°, which corresponds to geometrically equivalent positions of the component of the imaging device. In other words, image acquisitions at an angulation angle of γ are equivalent to such with an angulation angle of γ+180° or γ−180°. This angle degeneracy is nullified by the mapping to the target value range, which leads to an improved training result and/or faster convergence. Similarly, the approach is advantageous in its effect even when the output value range has a width of only 180° or less. Thus, an acquisition series having angulation angles in the range [symbol] [α [symbol] ₀, α₁] may be equivalent to an acquisition series having angulation angles in the range [symbol] [α [symbol] ₀+δ, α₁+δ] when the object in the latter case is rotated through −δ. Different results in the case of such equivalent scenarios are avoided by the MLM trained.

If the predicted measure is to be used further, for example, in order to estimate an actual motion of the object, it may be necessary to take into account the fact that the measure for the motion may not be predicted for one actual angulation angle, but if necessary for more than one, in particular two, angulation angles. This circumstance may be taken into account in the motion-compensated reconstruction as a function of the measure for the motion in that, for example, in an optimization method that is performed for the reconstruction, potentially multiple motion trajectories corresponding to the different angulation angles are taken into account. The resulting best variant may then be selected, for example.

In some embodiments, the width of the target value range is equal to 180°. Accordingly, the maximum geometrically unequivocal width is made use of such that the number of angulation angles that are mapped to the same effective angle according to the mapping rule may be kept as small as possible.

In some embodiments, the target value range is given by [0°, β₁] or by [−β₁, β₁]. In other words, β₀=0 or β₀=−β₁ applies.

In some embodiments, the width of the output value range is greater than the width of the target value range.

Accordingly, the output value range is mapped by the mapping rule to the smaller target value range having a width of 180° or less. This is particularly advantageous if the width of the output value range is greater than 180° and in particular less than or equal to 360°.

In such embodiments, the equivalence of angulation angles shifted through 180° is effectively bypassed by the mapping into the target value range. The training of the MLM is accordingly simplified or the training result improved since in particular apparently false predictions are not penalized by an increased value of the loss function. Assuming a training projection image having an angulation angle of 190° were corrupted by motion, whereas a training projection image having an angulation angle of 10° would not be corrupted by motion, In the reconstruction, and consequently in the three-dimensional training image data, the effect would not be distinguishable from a situation in which the training projection image having the angulation angle of 10° would be degraded by motion and the training projection image having the angulation angle of 190° not. In a conventional training method, the MLM would now be penalized if it were to predict a high measure for the motion for an angulation angle of 10°, which would negatively impact the convergence of the training and the reliability of the trained MLM. Both angulation angles may be mapped, for example, to an effective angle of 10°. The MLM only makes a prediction for the effective angle of 10° and not for an actual angulation angle. The complications outlined above may be avoided as a result.

The mapping rule may map a subsection of the output value range onto itself, in particular that each angle in the subsection is mapped to itself by the mapping rule. This may be particularly advantageous, for example, when the output value range has a width of more than 180°. In particular, the subsection may be identical to the target value range.

Given [0°, α₁] where α₁>180°, the mapping rule may provide that angles in the subsection [0°, 180°] are mapped to themselves and angles γ in the range]180°, α₁]γ to γ−180°.

In some embodiments, the width of the target value range is equal to 180° and the output value range is given by [0, α₁], where in particular α₁>180° applies.

In some embodiments, the output value range is given by [α₀, α₁], where α₀>0° and α₁>180° applies. The width of the output value range may be greater than 180°, but this is not necessarily the case.

In some embodiments, the motion state corresponds to a virtual spatial location of the object during a generation of the respective training projection image. A reference measure for the motion of the object is calculated as a function of the virtual spatial location. The value of the loss function is calculated as a function of a deviation between the reference measure and the predicted measure.

The virtual spatial location of the object may be defined in particular relative to the respective spatial location of the component of the imaging device or to another reference.

Accordingly, the MLM is trained in particular such that it may predict the reference measure as a measure for the motion as accurately as possible. The reference measure may correspond to the virtual spatial location of the object or be calculated based thereon. The choice of the reference measure may be dependent in particular on a potential further use of the measure for the motion predicted by the trained MLM following the training. If, for example, the predicted measure is to be used within the scope of an optimization method, for example, for estimating trajectories, certain measures may be advantageous as opposed to others, for example, with regard to the convergence of the optimization.

For example, the reference measure may correspond to a reprojection error in relation to a nominal spatial location of the object during the generation of the respective training projection image or be calculated as a function of the reprojection error. The nominal location may correspond to the location according to the desired trajectory.

In some embodiments, the three-dimensional training image data contains an entire volume reconstructed as a function of the plurality of training projection images and the virtual motion of the object, or the three-dimensional training image data includes the entire reconstructed volume.

This means that the maximum available three-dimensional information is made available to the MLM. The reliability and accuracy of the prediction by the MLM may be increased as a result.

In some embodiments, the three-dimensional training image data contains at least one three-dimensional first slice image corresponding to a first scanning direction through the entire volume reconstructed as a function of the plurality of training projection images and the virtual motion of the object. In particular, the training image data does not contain the entire reconstructed volume.

The three-dimensional first slice acquisition, also referred to as a slice image or a slice, is therefore a subvolume of the entire reconstructed volume. If the training image data contains two or more first slices corresponding to the first scanning direction, then the slices correspond to different axial positions along the first scanning direction. In this case, different first slices may also overlap, though this is not necessary. The first scanning direction may correspond to an axial, a sagittal, or a coronal cross-sectional view through the body of the patient.

If the at least one three-dimensional first slice image is used as input for the MLM instead of the entire reconstructed volume, the computational and storage overhead necessary for the training may be substantially reduced.

In some embodiments, the three-dimensional training image data contains the at least one three-dimensional first slice image corresponding to the first scanning direction and at least one three-dimensional second slice image corresponding to a second scanning direction through the entire volume reconstructed as a function of the plurality of training projection images and the virtual motion of the object. In particular, the training image data does not contain the entire reconstructed volume.

The second scanning direction is different from the first scanning direction, e.g., perpendicular to the first scanning direction. This enables further information to be made available as input to the MLM without the need to process the entire reconstructed volume. Accordingly, the reliability and accuracy of the prediction may be increased further with only a moderate increase in computing and storage requirements.

For example, the first scanning direction corresponds to the axial cross-sectional view and the second scanning direction corresponds to the sagittal or the coronal cross-sectional view. In another example, the first scanning direction corresponds to the sagittal cross-sectional view and the second scanning direction corresponds to the axial or the coronal cross-sectional view. In another example, the first scanning direction corresponds to the coronal cross-sectional view and the second scanning direction corresponds to the axial or the sagittal cross-sectional view.

In some embodiments, the three-dimensional training image data contains the at least one three-dimensional first slice image corresponding to the first scanning direction, the at least one three-dimensional second slice image corresponding to the second scanning direction and at least one three-dimensional third slice image corresponding to a third scanning direction through the entire volume reconstructed as a function of the plurality of training projection images and the virtual motion of the object. In particular, the training image data does not contain the entire reconstructed volume.

The third scanning direction is different from the first scanning direction and the second scanning direction, in particular, is perpendicular to the first scanning direction and the second scanning direction. This enables further information to be made available as input to the MLM without the need to process the entire reconstructed volume. Accordingly, the reliability and accuracy of the prediction may be increased further at the expense of a moderate increase in computing and storage requirements.

For example, the first scanning direction corresponds to the axial cross-sectional view, the second scanning direction corresponds to the sagittal cross-sectional view and the third scanning direction corresponds to the coronal cross-sectional view.

According to a further aspect, a computer-implemented method for motion detection is disclosed. In this method, a plurality of two-dimensional projection images representing an object are obtained, wherein each projection image of the plurality of projection images is assigned an angulation angle within an output value range. An effective angle in a target value range that is different from the output value range is calculated for each angulation angle of the plurality of training projection images by a predefined mapping rule, the width of the target value range amounting to a maximum of 180°. Three-dimensional image data is reconstructed as a function of the plurality of projection images. By applying an MLM, in particular a trained MLM, to the three-dimensional image data, a measure for a motion of the object is predicted for a predefined angle in the target value range, in particular for a motion of the object during the generation of the plurality of two-dimensional projection images.

Unless stated otherwise, all the acts of the computer-implemented method may be performed by a further data processing system including at least one further data processing device. In particular, the at least one further data processing device is configured or adapted for performing the acts of the computer-implemented method. For this purpose, the at least one further data processing device may store a further computer program containing commands that, when they are executed by the at least one further data processing device, cause the at least one further data processing device to perform the computer-implemented method. The computer-implemented method may also be implemented wholly or in part in hardware.

For the case in which the at least one further data processing device contains two or more further data processing devices, certain acts performed by the at least one further data processing device may also be understood in the sense that different further data processing devices perform different acts or different parts of an act. In particular it is not necessary for each further data processing device to perform the acts. In other words, the acts may be performed distributed over the two or more further data processing devices.

From each embodiment of the computer-implemented method for motion detection there is produced a corresponding embodiment of a method for motion detection that is not exclusively computer-implemented in that corresponding acts for generating the projection images are included.

The applying the MLM to the image data, and hence the prediction of the measure for the motion, may be performed for further angles in the target value range, for example, for all angles in the target value range. It is also possible to predict the measure for the motion for multiple, in particular all, angles in the target value range in one act, i.e., by one-time application of the MLM to the image data.

A known image reconstruction method may be used for reconstructing the three-dimensional image data, for example, a method in which the reconstruction is based on a motion model of a currently estimated movement of the object, as is explained, for example, in the publication by Preuhs et al. In particular, the motion model may be updated or adapted as a function of the predicted or generated motion data in order to facilitate an improved motion-compensated reconstruction. Alternatively, or in addition, the motion data or parts of the motion data may be used as a measure for motion artifacts or for the severity of the motion artifacts in a target function for the iterative optimization of the reconstruction.

In some embodiments, as a function of the predicted measure for the motion, a totality of all those projection images of the plurality of projection images whose effective angle corresponds to the predefined angle are identified or classified as degraded by motion, i.e., identified or classified in particular as motion-corrupted or non-motion-corrupted.

In particular, therefore, not necessarily individual projection images in particular are identified as degraded by motion, but only the totality of the projection images whose effective angle corresponds to the predefined angle.

For example, the three-dimensional image data may be determined based on the plurality of projection images and an estimated motion trajectory for the object or a desired motion trajectory for the object. In this regard reference is made to the publication by Preuhs et al. cited in the introduction.

In some embodiments, the measure for the motion is determined for all angles in the target value range, in particular according to a predefined discretization, by applying the MLM or by applying the MLM multiple times. A further estimated motion trajectory for the object is determined as a function of the measures determined for the motion.

Since it may happen that multiple projection images are assigned the same effective angle, it may be provided that multiple attempts or iterations in which the measure for the motion is assigned in each case to different projection images are performed in order to determine the further estimated motion trajectory.

For example, further three-dimensional image data may be reconstructed based on the further estimated motion trajectory and the plurality of two-dimensional projection images. As a result, the reconstruction may be improved, for example, iteratively, or the motion compensation during the reconstruction may be improved.

According to at least one embodiment of the computer-implemented method for motion detection, the MLM has been or is trained using a computer-implemented training method. In other words, the computer-implemented method for motion detection includes the acts of the computer-implemented training method.

According to a further aspect, a data processing system is disclosed, which is configured to perform a computer-implemented method.

According to a further aspect, a further data processing system is disclosed, which is configured to perform a computer-implemented training method.

In the present disclosure, the terms “data processing system” and “at least one data processing device” may be used interchangeably. As used herein, a data processing device may refer to a device that contains a processing circuit. The data processing device may therefore process data for performing computational operations. These may also include operations for performing indexed access to a data structure, for example, a look-up table (LUT), just like a data processing process implemented in hardware.

The data processing device may contain one or more computers, one or more microcontrollers, and/or one or more integrated circuits, for example, one or more application-specific integrated circuits (ASICs), one or more field-programmable gate arrays (FPGAs), and/or one or more systems on a chip (SoCs). The data processing device may also contain one or more processors, for example, one or more microprocessors, one or more central processing units (CPUs), one or more graphics processing units (GPUs), and/or one or more signal processors, in particular one or more digital signal processors (DSPs). The data processing device may also include a physical or a virtual network of computers or other of the cited units.

In different embodiments, the data processing device includes one or more hardware and/or software interfaces and/or one or more memory units.

A memory unit may be implemented as a volatile data memory, for example, as a dynamic random access memory (DRAM) or a static random access memory (SRAM), or as a nonvolatile data memory, for example, as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM).

According to a further aspect, an imaging apparatus is disclosed. The imaging apparatus includes an imaging device configured to generate a plurality of two-dimensional projection images that represent an object, each projection image of the plurality of projection images being assigned an angulation angle, in particular an angulation angle of the imaging device, within an output value range. The imaging apparatus includes a data processing system configured to perform a computer-implemented method for motion detection based on the plurality of two-dimensional projection images.

According to a further aspect, a computer program including commands is disclosed. When the commands are executed by a data processing system, the commands cause the data processing system to perform a computer-implemented training method.

According to a further aspect, a further computer program including further commands is disclosed. When the further commands are executed by a further data processing system, the further commands cause the further data processing system to perform a computer-implemented method for motion detection.

The commands and/or the further commands may be present, for example, in the form of program code. The program code may be provided, for example, as binary code or Assembler and/or as source code of a programming language, for example, C, and/or as a program script, for example, Python.

According to a further aspect, a computer-readable storage medium is disclosed, in particular a physical and/or nonvolatile or non-transitory computer-readable storage medium that stores a computer program and/or a further computer program.

The computer program, the further computer program, and the computer-readable storage medium are in each case computer program products containing the commands.

Above and in the following, the solution is described both in relation to the claimed systems and in relation to the claimed methods. Features, advantages, or alternative embodiments may be associated with the other claimed subject matters and vice versa. In other words, the claims and embodiments for the systems may be improved by features that are described or claimed in connection with the respective methods. In this case, the functional features of the method are implemented by physical units of the system.

Above and in the following, the solution is furthermore described in relation to methods and systems for motion detection as well as in relation to methods and systems for training an MLM. Features, advantages, or alternative embodiments may be associated with the other claimed subject matters and vice versa. In other words, claims and embodiments for training the MLM may be improved by features that are described or claimed in connection with the motion detection. In particular, the datasets used in the methods and systems may possess the same characteristics and features as the corresponding datasets that are used in the methods and systems for training the MLM, and the trained MLMs provided by the respective methods and systems may be used in the methods and systems for motion detection.

Further features and feature combinations of the disclosure are apparent from the figures and their description as well as from the claims. In particular, further embodiments of the disclosure do not necessarily have to contain all the features of one of the claims. Further embodiments of the disclosure may include features or feature combinations that are not cited in the claims.

The disclosure is explained in more detail below with reference to actual embodiments and associated schematic drawings. In the figures, like or functionally identical elements may be labeled with the same reference signs. The description of like or functionally identical elements may not necessarily be repeated in relation to different figures.

### BRIEF DESCRIPTION OF THE DRAWINGS

FIG. 1 shows a schematic view of an embodiment of an imaging apparatus.

FIG. 2 shows a schematic block diagram of an embodiment of a computer-implemented method for motion detection.

FIG. 3 shows a schematic block diagram of a further embodiment of a computer-implemented method for motion detection.

FIG. 4 shows a schematic block diagram of an embodiment of a computer-implemented training method.

FIG. 5 shows a schematic block diagram of a further embodiment of a computer-implemented training method.

FIG. 6 shows a schematic visualization of an embodiment of an output value range for angulation angles.

FIG. 7 shows a schematic visualization of an embodiment of a further output value range for angulation angles.

FIG. 8 shows a schematic visualization of an embodiment of a further output value range for angulation angles.

### DETAILED DESCRIPTION

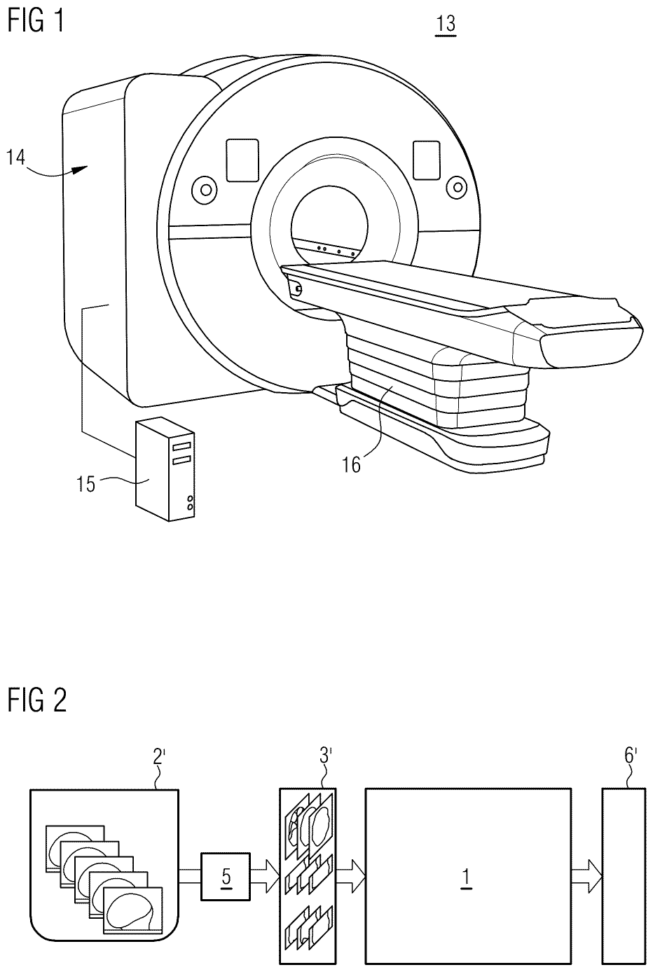

FIG. 1 schematically shows an embodiment of an imaging apparatus 13.

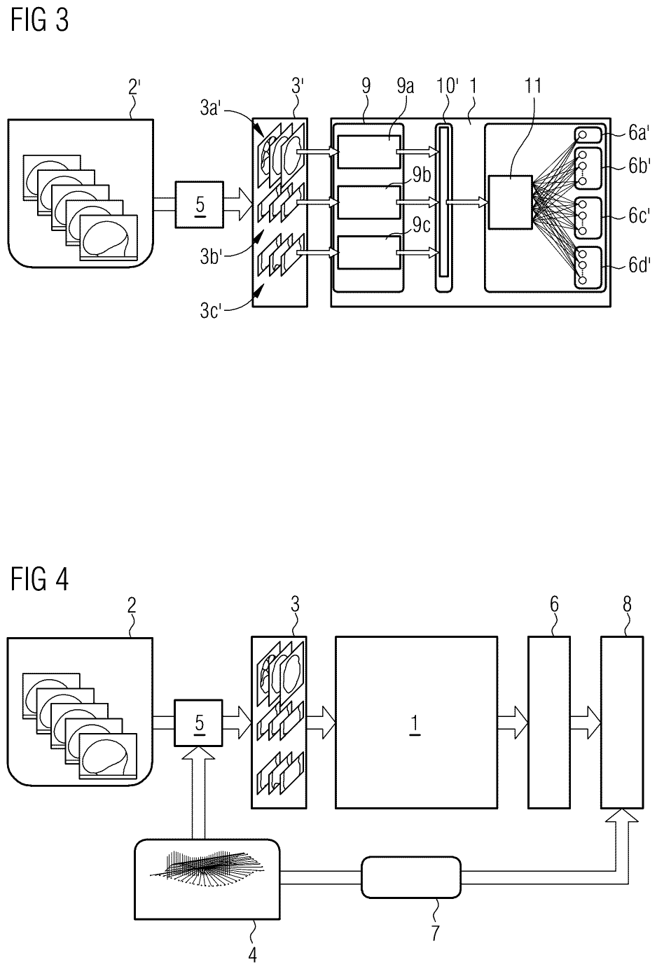

The imaging apparatus 13 includes an imaging device 14 configured to generate a plurality of two-dimensional projection images 2′ (see FIG. 2 and FIG. 3) representing an object, each of the projection images 2′ being assigned an angulation angle within an output value range. The imaging apparatus 13 includes a data processing system 15 configured to perform a computer-implemented method for motion detection based on the plurality of two-dimensional projection images 2′. The imaging apparatus 13 may include a patient couch 16 on which the object, in particular a patient, may be positioned for the purpose of generating the projection images 2″.

In the example of FIG. 1, the imaging device 14 is shown as a CT device. In other embodiments, however, the imaging device 14 may also be a different X-ray-based imaging device, in particular, a CBCT device and/or C-arm X-ray device, or an imaging device of a different modality capable of generating projection images at different angulation angles, for instance an MRT device. In the case of an X-ray-based imaging device such as a CT device or C-arm X-ray device, the imaging device 14 includes an X-ray source and an X-ray detector. The angulation angle is then given, for example, by an angle included by a connection line between X-ray source and X-ray detector in a plane standing perpendicular to a longitudinal axis of the imaging device 14 with a predefined reference direction, for example, a horizontal. The longitudinal direction may correspond to the longitudinal axis of the body of the patient. The angulation angle is therefore the corresponding angle in the transverse plane of the patient. In a CT device, the longitudinal direction may be parallel to the axis of rotation of the X-ray source and the X-ray detector, which may be referred to as the z-axis. With C-arm devices, the longitudinal direction may be defined analogously. In particular the angulation angle in the case of a C-arm device may be defined for a craniocaudal angle of 0°.

FIG. 2 shows a schematic block diagram of an embodiment of a computer-implemented method for motion detection, such as may be performed, for example, by the data processing system 15 of the imaging apparatus 13 from FIG. 1.

In this method, the plurality of two-dimensional projection images 2′ are obtained and an effective angle in a target value range that is different from the output value range is calculated for each angulation angle of the plurality of projection images 2′ by a predefined mapping rule, wherein the width of the target value range amounts to a maximum of 180°. Three-dimensional image data 3′ is reconstructed, in particular by a reconstruction algorithm 5, as a function of the plurality of projection images 2′. By applying a trained machine learning model, MLM, 1 to the three-dimensional image data 3′, a measure 6′ for a motion of the object is predicted for a predefined angle in the target value range. Optionally, this may be done for all angles in the target value range, the target value range then being discretized accordingly, for example, according to the angulation angles and the mapping rule. The fact that the output of the MLM 1 corresponds to a measure 6′ for the motion of the object follows from the corresponding upstream training of the MLM 1, for example, using a computer-implemented training method, as is discussed above and in the following also with reference to FIG. 4 and FIG. 5.

A nominal motion of the object or a currently estimated motion of the object may be taken as a basis for the reconstruction as a function of the plurality of projection images 2′. The nominal motion of the object or the currently estimated motion of the object may also be such that the object does not move relative to the patient couch 16. Nevertheless, in the case of a CT or C-arm device, a movement relative to the X-ray source also takes place then.

For example, the three-dimensional image data 3′ may be generated in the course of an iterative reconstruction method. In this process, in particular a measure for the motion-induced error susceptibility of the reconstructed image data 3′ is minimized. For example, the reprojection error may be used as such a measure, as described in the publication by Preuhs et al. cited in the introduction. Simplex methods or methods based on the Monte Carlo method, such as, say, CMA-ES (Covariance Matrix Adaptation Evolution Strategy), among others, come into consideration as suitable optimization methods.

The MLM 1 may be configured as an ANN, in particular as an ANN including a feature extraction stage and a regression stage, such as described in the cited publication by Preuhs et al. However, the ANN may also have a different, in particular CNN-based or transformer-based, architecture, such as the Swin transformer cited in the introduction.

The three-dimensional image data 3′ may contain an entire volume reconstructed as a function of the plurality of projection images 2′. In other embodiments, it is possible that instead of the entire reconstructed volume the three-dimensional image data 3′ contains a plurality of three-dimensional slice images corresponding to one or more scanning directions through the reconstructed volume, for example, corresponding to axial and/or sagittal and/or coronal sections.

FIG. 3 shows a schematic block diagram of a further embodiment of a computer-implemented method for motion detection, which is based on the method of FIG. 2.

In this example, the MLM 1 is constructed for instance as described in the publication by Preuhs et al. cited in the introduction. In this case, the input data 3′ includes axial slice images 3a′, sagittal slice images 3b′ and coronal slice images 3c′. The MLM 1 contains a Siamese triplet network 9 including three residual neural networks, RNNs, 9a, 9b, 9c, which each receive the axial slice images 3a′, the sagittal slice images 3b′ or the coronal slice images 3c′ as input. The features 10′ extracted in each case by the RNNs 9a, 9b, 9c, also referred to as attributes or embeddings, are then fed into a regression network 11, which may output a scalar output 6a′ and three vectorial outputs 6b′, 6c′, 6d′.

The RNNs 9a, 9b, 9c may be designed, for example, based on the ResNet-18, as described in Preuhs et al. The regression network may contain a 1×1 convolutional layer followed by a 1×1 global average pooling layer, as described in Preuhs et al.

For example, the output 6a′ corresponds to a reprojection error averaged over all angles in the target value range. The output 6b′ may contain a reprojection error for movements taking place in the respective projection plane (in-plane reprojection error) for each angle in the target value range. The output 6c′ may contain a reprojection error for movements taking place in a first plane perpendicular to the respective projection plane (out-of-plane reprojection error) for each angle in the target value range. For each angle in the target value range, the output 6d′ may contain a reprojection error for movements taking place in a second plane perpendicular to the respective projection plane and the first plane (out-of-plane reprojection error).

FIG. 4 shows a schematic block diagram of an embodiment of a computer-implemented training method for training an MLM 1 for motion detection, in particular an MLM 1, as has been described with reference to FIG. 2.

A plurality of two-dimensional training projection images 2 representing an object are obtained, each of the training projection images 2 being assigned an angulation angle within an output value range. The training projection images 2 may be generated, for example, by the imaging device 14, in which case it is provided that the object does not move relative to the patient table 16 during the generation or, in other words, the movement of the object during the generation of the training projection images 2 corresponds to a nominal motion defined solely by the movement of the components of the imaging device 14, in particular of the X-ray source.

Three-dimensional training image data 3 is reconstructed as a function of the plurality of training projection images 2 and a predefined virtual motion 4 of the object, each training projection image 2 being assigned a motion state according to the virtual motion 4. The virtual motion 4 corresponds to a simulated motion, in particular a rigid motion, which may be taken into account by the reconstruction algorithm 5 during the reconstruction, as described, for example, in Preuhs et al.

An effective angle in a target value range that is different from the output value range is calculated for each angulation angle of the plurality of training projection images 2 by a predefined mapping rule, a width of the target value range amounting to a maximum of 180°. A measure 6 for a motion of the object is predicted for a predefined angle in the target value range by applying the MLM 1 to the three-dimensional training image data 3. A value of a predefined loss function 8 is calculated as a function of the predicted measure 6 and the motion states of all those training projection images 2 whose effective angle corresponds to the predefined angle. The MLM 1 is updated as a function of the value of the loss function 8.

In particular, the motion state corresponds to a virtual spatial location of the object during the generation of the respective training projection image 2. A reference measure 7 for the movement of the object is calculated as a function of the virtual spatial location and the value of the loss function 8 is calculated as a function of a deviation between the reference measure 7 and the predicted measure 6.

FIG. 5 shows a schematic block diagram of a further embodiment of a computer-implemented training method for training an MLM 1 for motion detection, in particular an MLM 1, as has been described with reference to FIG. 3.

As described with reference to FIG. 3, the outputs 6a, 6b, 6c, 6d of the MLM 1 may correspond to the averaged reprojection error, the in-plane reprojection error and the out-of-plane reprojection errors and the reference measure 7 may contain corresponding reprojection errors of the virtual motion 4. The loss function 8 contains corresponding deviations 8a, 8b, 8c, 8d between the outputs 6a, 6b, 6c, 6d and the reprojection errors of the reference measure 7 or the virtual motion 4.

In particular, the functions cited in the equations (9) and (10) of the publication by Preuhs et al. may be used as the loss function 8, in which case the index i is replaced by the angles of the target value range.

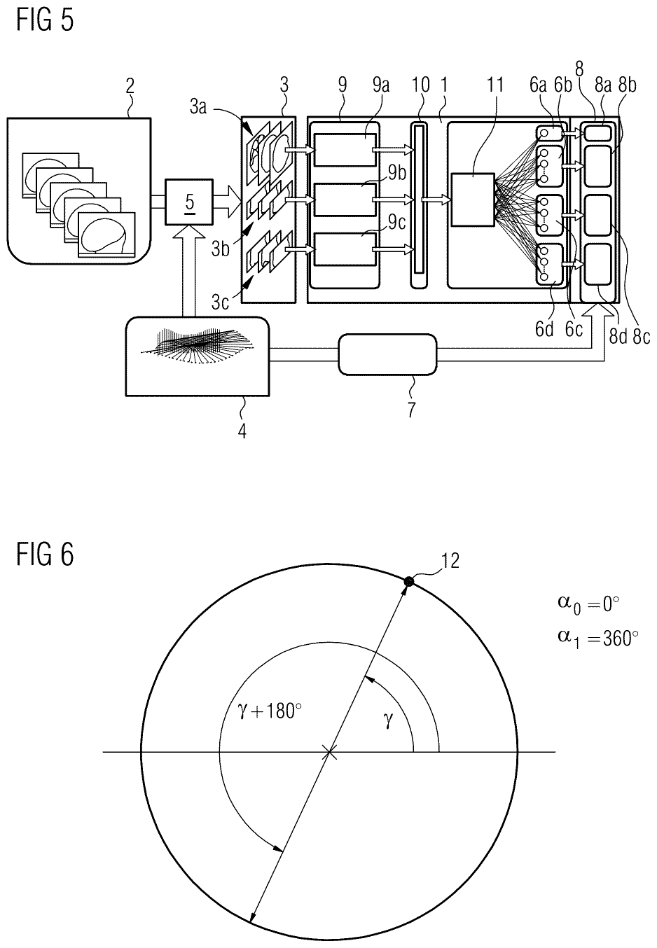

In figures FIG. 6 to FIG. 8, different case examples are visualized in which the approach is particularly advantageous. A position 12 of the X-ray source in the transverse plane and an angulation angle γ are shown in each case.

In FIG. 6, the output value range is given by [symbol] [α [symbol] _0, α_1 [where α₀=0° and α₁=360°. With conventional approaches, this may lead to problems in the training of the MLM since the MLM cannot distinguish, from the three-dimensionally reconstructed data, between a movement at the angulation angle γ and a movement at the angulation angle γ+180°. This is avoided by the limiting to the target value range having a width of max. 180°, in particular a width of 180°.

The mapping function may be such that:

$$γ ↦ γ , if γ ∈ [ 0 ° , 180 ° ] ,$$

$$γ ↦ γ - 180 ° , if γ ∈ [ 180 ° , 360 ° ] .$$

However, other functions having a corresponding value range may also be used.

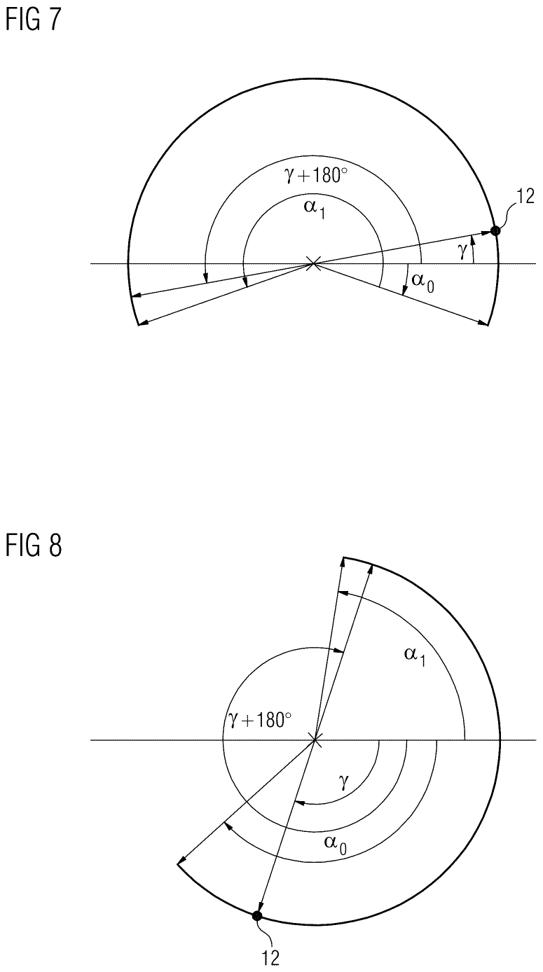

Similarly to FIG. 6, with conventional approaches, the same problems also occur in scenarios like that shown in FIG. 7. In this case, the output value range is given by [symbol] [α [symbol] ₀, α₁] where −90°<α₀<0° and 180°<α₁<270°, in particular α₁=180°−α₀. For example, the output value range may be given by [−20°, 200°] such that the width of the output value range amounts to 220°.

An effective equivalence of angulation angle γ and angulation angle γ+180° is also present here if α₀<γ<0° or 180°<γ<α₁.

In FIG. 8, a similar situation is illustrated as in FIG. 7, except that here the output value range is not shifted symmetrically around 90° but correspondingly in the clockwise direction. The advantages of the disclosure also manifest themselves in such constellations. Furthermore, the disclosure also has advantages here if the output value range has a width of only 180° or less. Thanks to the mapping of the angulation angles to the target value range, the MLM 1 sees consistent input data, even in the case of different shifts of the output value range, which may be necessary, for example, for adapting to the position of the object on the patient couch 16.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for training a machine learning model (MLM) for motion detection based on three-dimensional image data, the computer-implemented method comprising:
obtaining a plurality of two-dimensional training projection images that represent an object, wherein each training projection image of the plurality of two-dimensional training projection images is assigned an angulation angle within an output value range;
reconstructing three-dimensional training image data as a function of the plurality of two-dimensional training projection images and a predefined virtual motion of the object, wherein each training projection image is assigned a motion state according to the predefined virtual motion;
calculating an effective angle in a target value range that is different from the output value range for each angulation angle of the plurality of two-dimensional training projection images by a predefined mapping rule, wherein a width of the target value range amounts to a maximum of 180°;
predicting a measure for a motion of the object by applying the MLM to the three-dimensional training image data for a predefined angle in the target value range;
calculating a value of a predefined loss function as a function of the predicted measure and the motion state of each training projection image of the plurality of two-dimensional training projection images having an effective angle that corresponds to the predefined angle; and
updating the MLM as a function of the value of the predefined loss function.

2. The computer-implemented method of claim 1, wherein the width of the target value range is equal to 180°, and/or
wherein the target value range is given by [0°, β₁] or by [−β₁, β₁].

3. The computer-implemented method of claim 1, wherein the motion state corresponds to a virtual spatial location of the object during a generation of the respective training projection image,
wherein a reference measure for the motion of the object is calculated as a function of the virtual spatial location, and
wherein the value of the loss function is calculated as a function of a deviation between the reference measure and the predicted measure.

4. The computer-implemented method of claim 3, wherein the reference measure corresponds to a reprojection error in respect of a nominal spatial location of the object during the generation of the respective training projection image, or
wherein the reference measure is calculated as a function of the reprojection error.

5. The computer-implemented method of claim 1, wherein the three-dimensional training image data comprises an entire volume reconstructed as a function of the plurality of two-dimensional training projection images and the virtual motion of the object, or
wherein the three-dimensional training image data comprises at least one three-dimensional slice image corresponding to a scanning direction through an entire volume reconstructed as a function of the plurality of two-dimensional training projection images and the virtual motion of the object.

6. The computer-implemented method of claim 1, wherein the width of the output value range is greater than the width of the target value range.

7. The computer-implemented method of claim 6, wherein the mapping rule maps a subrange of the output value range to itself.

8. The computer-implemented method of claim 1, wherein the width of the output value range is greater than 180° and less than or equal to 360°.

9. The computer-implemented method of claim 8, wherein the width of the target value range is equal to 180°, and
wherein the output value range is given by [0, α₁].

10. The computer-implemented method of claim 1, wherein the output value range is given by [α₀, α₁], where α₀>0° and α₁>180°.

11. A computer-implemented method for motion detection, the computer-implemented method comprising:
obtaining a plurality of two-dimensional projection images that represent an object wherein each projection image of the plurality of two-dimensional projection images is assigned an angulation angle within an output value range;
calculating an effective angle in a target value range that is different from the output value range for each angulation angle of the plurality of two-dimensional projection images by a predefined mapping rule, wherein a width of the target value range amounts to a maximum of 180°;
reconstructing three-dimensional image data as a function of the plurality of projection images; and
predicting a measure for a motion of the object by applying a machine learning model (MLM) to the three-dimensional image data for a predefined angle in the target value range.

12. The computer-implemented method of claim 11, further comprising, as a function of the predicted measure:
identifying, as degraded by motion, a totality of projection images of the plurality of two-dimensional projection images whose effective angle corresponds to the predefined angle.

13. A system comprising:
one or more processors configured to:
obtain a plurality of two-dimensional training projection images that represent an object, wherein each training projection image of the plurality of two-dimensional training projection images is assigned an angulation angle within an output value range;
reconstruct three-dimensional training image data as a function of the plurality of two-dimensional training projection images and a predefined virtual motion of the object, wherein each training projection image is assigned a motion state according to the predefined virtual motion;
calculate an effective angle in a target value range that is different from the output value range for each angulation angle of the plurality of two-dimensional training projection images by a predefined mapping rule, wherein a width of the target value range amounts to a maximum of 180°;
predict a measure for a motion of the object by applying machine learning model (MLM) to the three-dimensional training image data for a predefined angle in the target value range;
calculate a value of a predefined loss function as a function of the predicted measure and the motion state of each training projection image of the plurality of two-dimensional training projection images having an effective angle that corresponds to the predefined angle; and
update the MLM as a function of the value of the predefined loss function.

14. The system of claim 13, further comprising:
an imaging device configured to generate the plurality of two-dimensional projection images representing the object.
